(19) United States
(12) Patent Application Publication
(10) Pub. No.: US 2013/0045135 A1
(43) Pub. Date: Feb. 21, 2013

(54) Grommet Device with Pull-Tab and Associated Methods Thereof

(71) Applicant: Kraig Herman Allen, Leesburg, IN (US)

(72) Inventor: Kraig Herman Allen, Leesburg, IN (US)

(73) Assignee: SYMMETRY MEDICAL MANUFACTURING, INC., Warsaw, IN (US)

(21) Appl. No.: 13/587,526

(22) Filed: Aug. 16, 2012

Related U.S. Application Data
(60) Provisional application No. 61/524,533, filed on Aug. 17, 2011.

(51) Int. Cl. F16L 5/02; A61L 2/16

(52) U.S. Cl. 422/28; 16/2.1; 422/292

(57) ABSTRACT

A grommet device for use with a medical instrument sterilization tray is provided. The grommet device includes a base structure having a base surface. A top structure has a top surface, wherein the top structure is connected to the base structure with a middle portion. An aperture is connected between the base surface and the top surface, and positioned within the base structure, the top structure, and the middle portion. A pull-tab is connected to the top structure.

### CROSS REFERENCE TO RELATED APPLICATION

This application claims benefit of U.S. Provisional Application Ser. No. 61/524,533 entitled “Grommet Device with Pull-Tab” and filed Aug. 17, 2011, the entire disclosure of which is incorporated herein by reference.

### FIELD OF THE DISCLOSURE

The present disclosure is generally related to grommets and more particularly is related to a grommet device with pull-tab.

### BACKGROUND OF THE DISCLOSURE

Within the medical industry, there is a need for holding a variety of medical instruments for various purposes. For example, a surgeon needs to be able to access medical instruments for surgery quickly, a dentist needs to be able to access his or her dental tools, and virtually all medical instruments must be placed within a holder during a sterilization process. Conventional holding containers may include a variety of bases holding insertable trays that have specifically-designed areas for holding specific tools. However, with smaller tools, such as small dental tools, it is frequently inefficient to store them in these containers, since they're prone to being moved around and jostled as the container is moved. This may result in a grouping of smaller tools in one area, which means that the surgeon or medical staff member must sift through the grouping to locate a specific tool.

Conventionally, medical instruments are often held in containers or trays with holes and grommets. The grommets may be positioned within the holes and provide a secure interface between the medical instrument and the hole within the container or tray. Often, the grommets are sized to match a certain shaft size of various medical instruments, and a container or tray may include a variety of different sized grommets to allow for holding a variety of medical instruments. Because of the risk of harboring bacteria and other contaminants, the grommets are permanently installed within the holes, with the surfaces of the grommets forming tight seals with the container or the tray. This may prevent bacterial from becoming lodge within cracks, crevices or other areas, which may prevent complete sterilization of the medical tool. However, users often try and remove the grommets when they become damaged, or when they desire to reposition the grommet in a new location. This removal of the grommet may result in damage to the grommet structure itself, as well as present additional areas for harboring bacterial.

Thus, a heretofore unaddressed need exists in the industry to address the aforementioned deficiencies and inadequacies.

### SUMMARY OF THE DISCLOSURE

Embodiments of the present disclosure provide a system and method for a grommet device. Briefly described, in architecture, one embodiment of the system, among others, can be implemented as follows. The grommet device includes a base structure having a base surface. A top structure has a top surface, wherein the top structure connected to the base structure with a middle portion. An aperture is connected between the base surface and the top surface, and positioned within the base structure, the top structure, and the middle portion. A pull-tab is connected to the top structure.

The present disclosure can also be viewed as providing a medical instrument sterilization system. Briefly described, in architecture, one embodiment of the system, among others, can be implemented as follows. A sterilization tray has at least one hole formed therein. A grommet device is removably positioned within the at least one hole, wherein the grommet device has an aperture formed at a substantially center point of the grommet device. A pull-tab is integrally affixed to the grommet device, wherein the pull-tab is positioned lateral to the at least one hole and above the at least one hole formed within the sterilization tray.

The present disclosure can also be viewed as providing methods of using a grommet device within a sterilization tray. In this regard, one embodiment of such a method, among others, can be broadly summarized by the following steps: inserting the grommet device into a hole within the sterilization tray; placing a medical instrument within an aperture in the grommet device; subjecting the medical instrument, grommet device, and sterilization tray to at least one sterilization process; removing the medical instrument from the aperture; and removing the grommet device from the hole within the sterilization tray by biasing a pull-tab integrally affixed to the grommet device.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a cross-sectional illustration of a grommet device, in accordance with a first exemplary embodiment of the present disclosure.

FIG. 2 is a cross-sectional view illustration of the grommet device, in accordance with the first exemplary embodiment of the present disclosure.

FIG. 3 is a top view illustration of the grommet device, in accordance with the first exemplary embodiment of the present disclosure.

FIG. 4 is a cross-sectional view illustration of the grommet device in a partially removed position, in accordance with the first exemplary embodiment of the present disclosure.

FIG. 5 is a top view illustration of the grommet device, in accordance with a second exemplary embodiment of the present disclosure.

FIG. 6 is a cross-sectional view illustration of the grommet device, in accordance with the second exemplary embodiment of the present disclosure.

FIG. 7 is a flowchart illustrating a method of constructing a grommet device, in accordance with a third exemplary embodiment of the disclosure.

### DETAILED DESCRIPTION

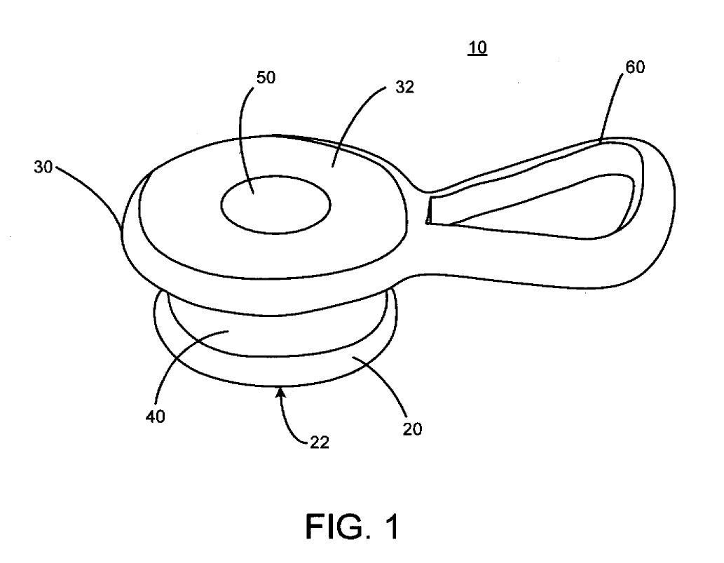

FIG. 1 is a cross-sectional illustration of a grommet device 10, in accordance with a first exemplary embodiment of the present disclosure. The grommet device 10, which may be referred to simply as ‘device 10’ or ‘grommet 10’, includes a base structure 20 having a base surface 22. A top structure 30 has a top surface 32, wherein the top structure 30 connected to the base structure 20 with a middle portion 40. An aperture 50 is connected between the base surface 22 and the top surface 32, and positioned within the base structure 20, the top structure 30, and the middle portion 40. A pull-tab 60 is connected to the top structure 30.

The device 10 may be used with medical tool holding structures, such as sterilization trays used for holding medical instruments during a sterilization process. Accordingly, the device 10 may be used in any industry utilizing medical tools, such as tools, instruments, or any other type of implement used for surgical procedures, operations, or other medical procedures. For example, the device 10 may be used to hold medical instruments in surgical environments before, during and/or after a surgical procedure, or a medical instrument sterilization process. Similarly, the device 10 may be used with dental instruments for dental operations, routine cleanings, or for any other use. Other settings and uses within the medical field are also envisioned, all of which are considered within the scope of the present disclosure.

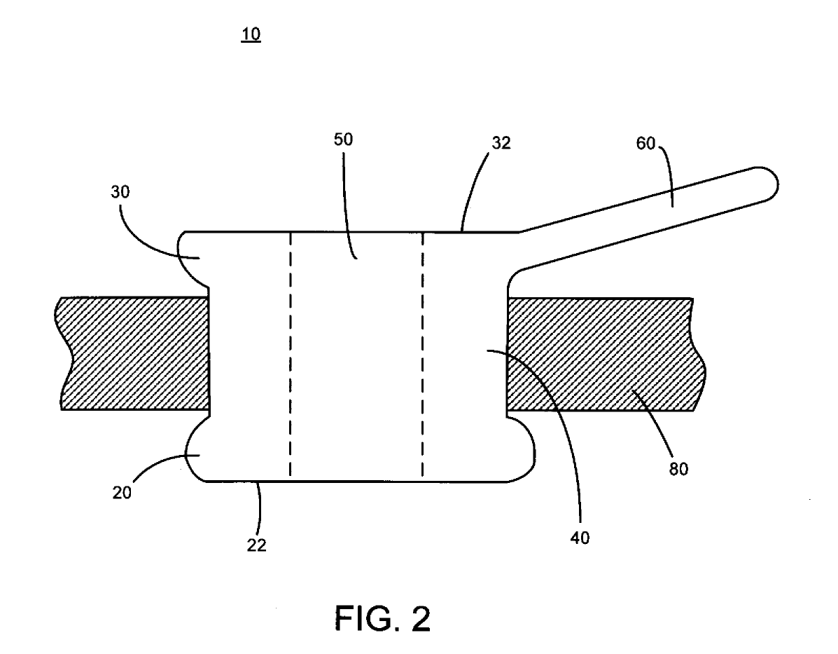

FIG. 2 is a cross-sectional view illustration of the grommet device 10, in accordance with the first exemplary embodiment of the present disclosure. As is shown in FIGS. 1-2, the base structure 20 of the device may be configured to be placed on one side of a grommet holding structure 80, which may be an opening, hole, or aperture within a medical sterilization tray or other, similar structure. The base structure 20 may be sized slightly larger than the grommet holding structure 80, thereby allowing the base structure 20 to be biased into position. For example, many sterilization trays include a plurality of holes for holding medical instruments. To secure the medical instrument properly during a medical sterilization process, the device 10 may be inserted into the hole by pushing the base structure 20 through the hole until the base structure 20 is located on one side of the tray, and the top structure 30 is located on an opposing side of the tray. Accordingly, the hole may be the grommet-holding structure 80. In this position, the base surface 22 may be positioned interior of, or below the medical sterilization tray. In other words, the plane of the base surface 22 may be substantially parallel to the plane of the sterilization tray, but not co-planar to the plane of the sterilization tray.

The top structure 30 may be sized similar to the base structure 20, in that the top structure 30 is sized larger than the grommet-holding structure 80. This may prevent the device 10 from slipping or moving out of position within the grommet-holding structure 80 accidentally. The top structure 30 may also be oriented such that the plane of the top surface 32 is substantially parallel to the plane of the sterilization tray or other structure that the grommet-holding structure 80 is positioned in, but not co-planar to the sterilization tray. Accordingly, the base and top structures 20, 30 may have any size exterior diameters, thicknesses, or other dimensions. Similarly, the overall dimensions of the device 10, including the overall thickness and external diameter may have any size.

The middle portion 40 may integrally connect the base structure 20 to the top structure 30. The middle portion 40 may commonly have a substantially cylindrical exterior shape that is configured to be positioned within the grommet-holding structure 80. For example, as is shown in FIG. 2, the middle portion 40 may be positioned abutting the grommet-holding structure 80, whereby the base and top structures 20, 30 are positioned below and above the grommet-holding structure 80, respectively. In order to allow the device 10 to be positioned and retained within the grommet-holding structure 80, the width of the middle portion 40 should be less than the width of the top structure 30 and the width of the base structure 20, as is shown in FIG. 2. The width of the base structure 20 and the top structure 30, however, should be larger than the diameter of the hole within the grommet-holding structure 80 to prevent the device 10 from inadvertently slipping out of the hole. When the device 10 is positioned within the hold, the exterior surface of the middle portion 40 should contact and engage with an interior surface of the hole.

The aperture 50 may be positioned within the device 10 connected between the top surface 32 with the base surface 22. In other words, the aperture 50 is a cut-out of material or hole that is positioned within the device 10, commonly aligned along a central axis of the cylindrical shape of the device 10. As the aperture 50 is used to hold medical instruments within the grommet-holding structure 80, the aperture 50 should have at least two open ends, and should not be a cavity with only one open end. The central axis may run through a radial center point of the grommet-holding structure 80, or may be positioned off-center, as various designs may dictate. The aperture 50 may be sized to hold any type of medical instrument, and thus, may have any size diameter.

Positioned proximate to the top structure 30 is the pull-tab 60. Commonly, the pull-tab 60 is integral with, affixed to, or integrally affixed to the top structure 30, such that a force exerted on the pull-tab 60 transfers to the top structure 30, which of course, is transferred throughout the device 10. The pull tab 60 may be connected to any portion of the top structure 30, such as an edge of the top structure 30, and the pull-tab 60 may be positioned at an angle with respect to the top surface 32 of the top structure 30 The Pull-tab 60 may have a variety of shapes and designs that allow it to be conveniently grasped by a used. For example, as is shown in FIG. 1, the pull-tab 60 may have an open interior portion that allows for convenient holding of the pull-tab 60.

The device 10 may be a substantially cylindrical structure around the base structure 20, the top structure 30, the middle portion 40, and the pull-tab 60, all of which may be integrally connected. For example, the top structure 30, the base structure 20, the middle portion 40, and the pull-tab 50 may be integrally molded together as a unitary structure. Commonly, the device 10 may be constructed from a rubber or silicon material that is substantially resistant to degradation from use and from sterilization environments. Constructing the device 10 from rubber or silicone may also allow the device 10 to have sufficient flexibility, thereby allowing it to retain its shape while positioned within the medical instrument tray, but allowing it to be flexible enough to be removed from the medical instrument tray. Within the medical industry, medical instruments are often sterilized in autoclaves, which utilize high temperatures, high pressures, moisture, and/or chemicals to sterilize a medical instrument, so the material that the device 10 is constructed from should be capable of withstanding the sterilization environment.

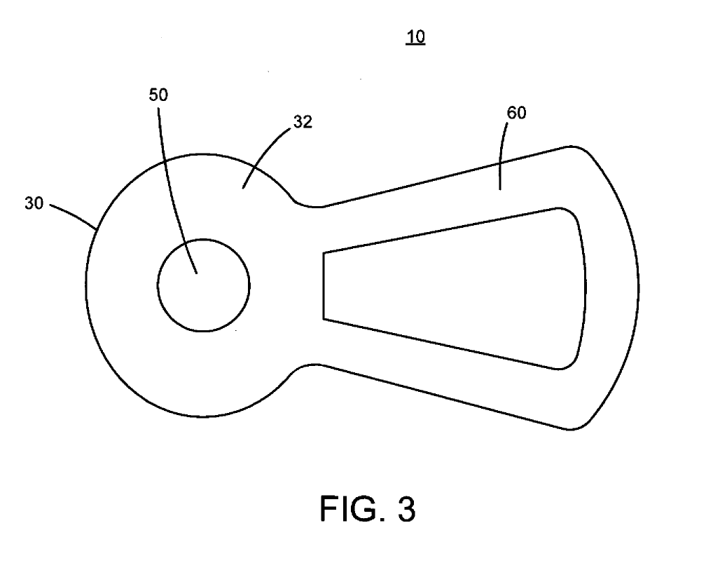

FIG. 3 is a top view illustration of the grommet device 10, in accordance with the first exemplary embodiment of the present disclosure. As is shown, the pull-tab 60 may be integrally connected with the top structure 30 of the device 10. The top surface 32, which encircles the aperature, may be substantially planar with a top surface of the pull-tab 60, at least at a beginning point of the pull-tab 60. The open interior of the pull-tab 60 is best shown in FIG. 3. As can be seen, the open interior may be size to allow a user's finger to penetrate within the interior, thereby allowing the user to apply a force to the pull-tab 60. Although the open interior may include any shape, size or configuration, a pull-tab 60 does not necessarily require an open interior. For example, the pull-tab 60 may include other textural features, such as ridges, bumps, or textures to allow a user to sufficiently grip the pull-tab 60 to remove the device 10.

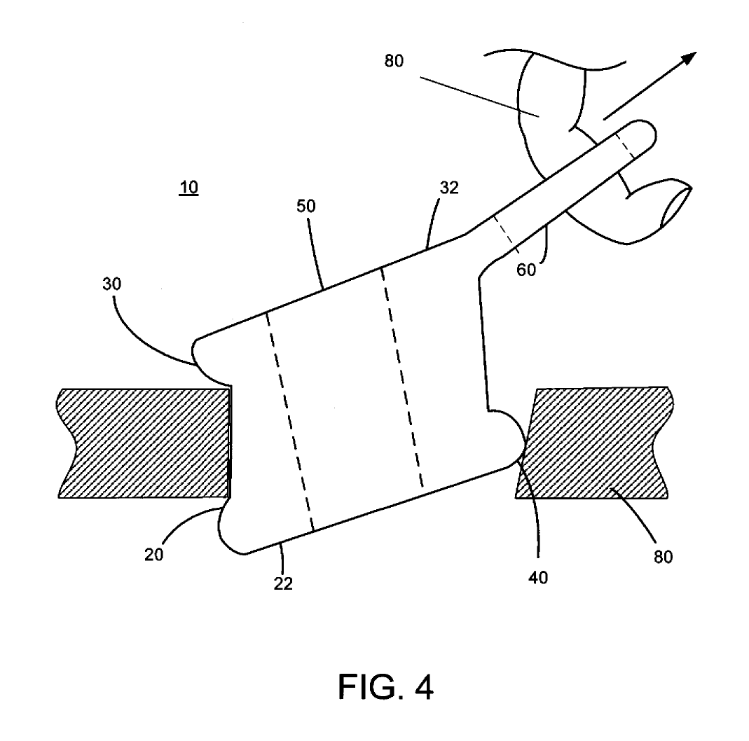

FIG. 4 is a cross-sectional view illustration of the grommet device 10 in a partially removed position, in accordance with the first exemplary embodiment of the present disclosure. FIG. 4 includes the base structure 20 having a base surface 22, the top structure 30 having a top surface, and the middle portion 40, as previously discussed. Further, FIG. 4 depicts the device 10 in the process of being removed from the grommet-holding structure 80. The pull-tab 60 allows a user to remove the device 10 from an installed position within the grommet-holding structure 80. As the grommet device 10 is constructed from a rubber, silicon, or other malleable material, it will allow for compression and manipulation of the base structure 20, such that it will fit through the hole in the grommet-holding structure 80, yet be rigid enough to retain its shape while the it is positioned within the hole of the grommet-holding structure 80. While not illustrated, the top structure 30, middle portion 40, and base structure 20 may bow while pulled through the grommet-holding structure 80 as needed.

Accordingly, when a device 10 needs to be removed or replaced, such as due to degradation, cleaning, or changing positions of the various devices, the pull-tab 60 may be grasped by a user and pulled away from the grommet-holding structure 80. The user may place a finger 8 through the open interior portion of the pull-tab 60 and bias the pull-tab 60 angularly away from the hole of the grommet-holding structure 80. Generally, the pull-tab 60 may be pulled at an angle away from the grommet-holding structure 80, such as a 45° angle, which allows the base structure 20 to slip through the grommet-holding structure 80 and free the device 10 from contact with the grommet-holding structure 80. To assist with pulling the pull-tab 60 at an angle, the pull-tab 60 may be oriented to have a natural angle from the position of the aperture 50, as is shown best in FIG. 2.

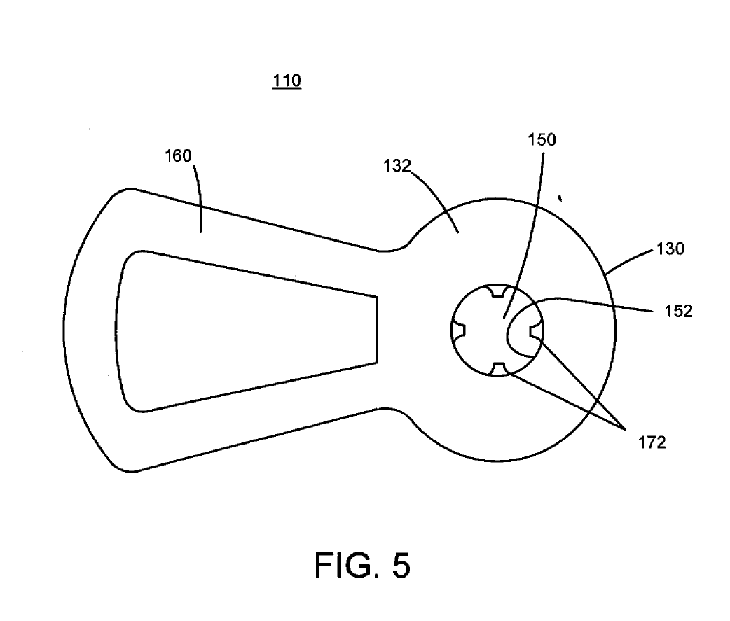

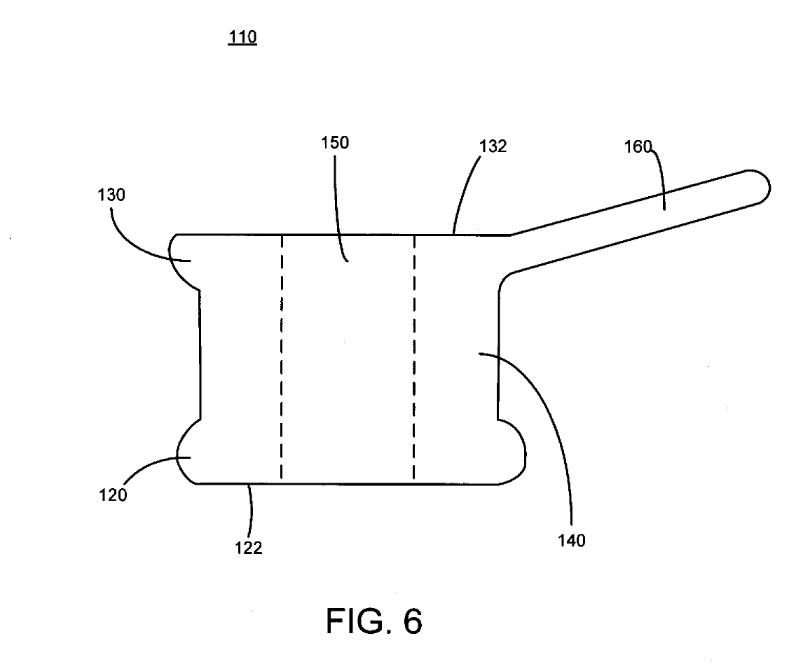

FIG. 5 is a top view illustration of the grommet device 110, in accordance with a second exemplary embodiment of the present disclosure. FIG. 6 is a cross-sectional view illustration of the grommet device 110, in accordance with the second exemplary embodiment of the present disclosure. The grommet device 110, which may be referred to simply as ‘device 110’ or ‘grommet 110’, may include any of the structures, features, or functions disclosed with respect to the other embodiments of this disclosure. The device 110 includes a base structure 120 having a base surface 122. A top structure 130 has a top surface 132, wherein the top structure 130 connected to the base structure 120 with a middle portion 140. An aperture 150 is connected between the base surface 122 and the top surface 132, and positioned within the base structure 120, the top structure 130, and the middle portion 140. A pull-tab 160 is connected to the top structure 130.

The device 110 is substantially similar to the device 10 of FIGS. 1-4. However, the second exemplary embodiment includes a plurality of protrusions 170 positioned on an inner wall 152 of the aperture 150 and extending into the aperture 150. The protrusions 170 may assist with holding a medical instrument, by interfacing with the shaft of the medical instrument and retaining it within the device 110. The protrusions 170 allow for sterilization material and gasses to flow around the portion of the medical instrument that is positioned within the aperture 150 during the sterilization process. The protrusion 170 may include any type of inward-facing tabs, formed from any type of material. Commonly, the protrusions 170 will be formed from the same material as the device 110 and be integral with at least one of the base structure, top structure 130, or middle portion.

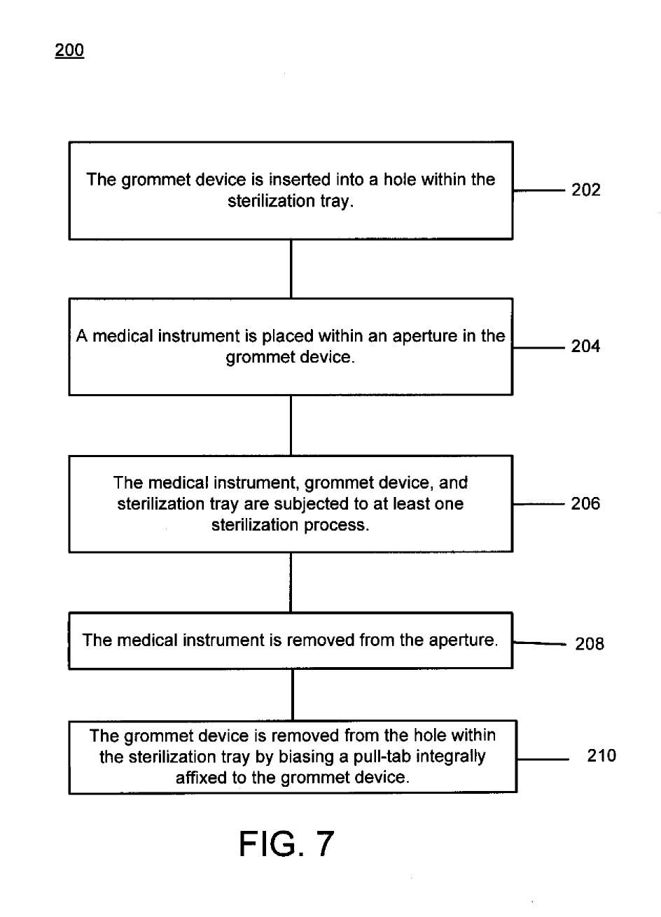

FIG. 7 is a flowchart 200 illustrating a method of using a grommet device within a sterilization tray, in accordance with a third exemplary embodiment of the disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As is shown by block 202, the grommet device is inserted into a hole within the sterilization tray. A medical instrument is placed within an aperture in the grommet device (block 204). The medical instrument, grommet device, and sterilization tray are subjected to at least one sterilization process (block 206). The medical instrument is removed from the aperture (block 208). The grommet device is removed from the hole within the sterilization tray by biasing a pull-tab integrally affixed to the grommet device (block 210).

The method may include any number of additional steps, processes, or functions, including any of the steps, processes, and functions disclosed with respect to FIGS. 1-5 herein. For example, the method may include the step of removing the grommet device from the hole within the sterilization tray by biasing the pull-tab angularly away from the hole. The angle that the pull-tab is biased at may be any angle, commonly around 45° from the planar surface of the sterilization tray. However, the angle that the pull-tab is biased at may largely depend on the size of the grommet device, the tolerance and fit between the grommet device and the hole, and the material that the grommet device is constructed from. When the grommet device is being removed from the hole within the sterilization tray, the base structure of the grommet device will be pulled through the hole (as is depicted in FIG. 4). Once the grommet device is removed, it may be discarded or sterilized for future use.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

What is claimed is:

1. A grommet device comprising:
a base structure having a base surface;
a top structure having a top surface, the top structure connected to the base structure with a middle portion, wherein an aperture is connected between the base surface and the top surface, and positioned within the base structure, the top structure, and the middle portion; and
a pull-tab connected to the top structure.

2. The grommet device of claim 1, wherein the pull-tab is connected to an outer edge of the top structure.

3. The grommet device of claim 1, wherein the pull-tab further comprises an open interior portion.

4. The grommet device of claim 1, wherein the pull-tab is aligned at an angle with respect to the top surface of the top structure.

5. The grommet device of claim 1, wherein the top structure, the base structure, the middle portion, and the pull-tab are integrally molded together.

6. The grommet device of claim 1, further comprising at least one protrusion positioned on an inner wall of the aperture and extending into the aperture.

7. The grommet device of claim 1, wherein the aperture is formed about a substantially central axis, wherein the substantially central axis is positioned substantially at a radial center point of the top structure, the base structure, and the middle portion.

8. The grommet device of claim 1, wherein a width of the middle portion is less than a width of the top structure and the width of the base structure.

9. The grommet device of claim 1, wherein the middle portion has a substantially cylindrical exterior shape.

10. The grommet device of claim 1, wherein the base structure, the top structure, the middle portion, and the pull-tab are constructed from at least one of: a rubber material and a silicone material.

11. A medical instrument sterilization system comprising:
a sterilization tray having at least one hole formed therein;
a grommet device removably positioned within the at least one hole, the grommet device having an aperture formed at a substantially center point of the grommet device; and
a pull-tab integrally affixed to the grommet device, wherein the pull-tab is positioned lateral to the at least one hole and above the at least one hole formed within the sterilization tray.

12. The medical instrument sterilization system of claim 11, wherein the grommet device further comprises:
a base structure having a base surface;
a top structure having a top surface; and
a middle portion connected between the top structure and the base structure.

13. The medical instrument sterilization system of claim 12, wherein a diameter of the base structure and a diameter of the top structure are both larger than a diameter of the at least one hole.

14. The medical instrument sterilization system of claim 12, wherein an exterior surface of the middle portion is sized to engage with an interior surface of the at least one hole when the grommet device is positioned within the at least one hole.

15. The medical instrument sterilization system of claim 12, wherein a plane of the base surface is substantially parallel to a plane of an underside of the sterilization tray, and wherein the plane of the base surface is not co-planar to the plane of the underside of the sterilization tray.

16. The medical instrument sterilization system of claim 11, wherein the pull-tab further comprises an open interior portion.

17. A method of using a grommet device within a sterilization tray, the method comprising the steps of:
inserting the grommet device into a hole within the sterilization tray;
placing a medical instrument within an aperture in the grommet device;
subjecting the medical instrument, grommet device, and sterilization tray to at least one sterilization process;
removing the medical instrument from the aperture; and
removing the grommet device from the hole within the sterilization tray by biasing a pull-tab integrally affixed to the grommet device.

18. The method of claim 17, wherein the step of removing the grommet device from the hole within the sterilization tray further comprises biasing the pull-tab angularly away from the hole.

19. The method of claim 17, wherein the step of removing the grommet device from the hole within the sterilization tray further comprises pulling a base structure of the grommet device through the hole.

20. The method of claim 17, further comprising the step of subjecting the removed grommet device to a sterilization process.
